(19) United States
(12) Patent Application Publication
(10) Pub. No.: US 2002/0197600 A1
(43) Pub. Date: Dec. 26, 2002

(54) Method for determining the presence of infection in an individual

(76) Inventors: Theodore E. Maione, Wakefield, MA (US); Yongwu Yang, Belmont, MA (US)

Correspondence Address: PENNIE & EDMONDS LLP, 1667 K STREET NW, SUITE 1000, WASHINGTON, DC, 20006

(21) Appl. No.: 10/144,778

(22) Filed: May 15, 2002

Related U.S. Application Data
(60) Provisional application No. 60/290,654, filed on May 15, 2001.

(51) Int. Cl. C12Q 1/70; G06F 19/00; G01N 33/48; G01N 33/50

(52) U.S. Cl. 435/5; 702/19

(57) ABSTRACT

The present invention relates to a method for determining whether a first biological sample is infected with one of HIV and hepatitis. The first biological sample is obtained from a first mammal. A first and second parameter are compared. A difference between the parameters is indicative of whether the first sample is infected with at least one of HIV and hepatitis. The first parameter is obtained from a fluorescence emission spectrum that results from a first fraction of the first biological sample, the first biological sample having been fractionated into the first fraction and a second fraction. The first fraction includes a greater relative amount of lipoprotein than the second fraction. The second parameter is obtained from a fluorescence emission resulting from a second biological sample obtained from a second, different mammal. The first and second fluorescence emissions are essentially free of fluorescence emission resulting from substances not-native to the first and second mammals, respectively.

### FIELD OF THE INVENTION

The present invention relates to a method for determining whether an individual is infected with a virus.

### BACKGROUND

Determination of whether an individual is infected with one or more viruses, such as Hepatitis and HIV viruses, reduces the probability that recipients of donated blood products become infected and helps the diagnosis and treatment of the infected individual. Known techniques for analyzing biological samples, such as blood, by spectroscopic methods seek to measure spectroscopic signals, such as fluorescence, resulting from one or more normative compounds that have been added to the biological sample. Spectroscopic techniques that do not rely on the detection of non-native compounds to determine whether a sample was obtained from an infected or infection-free individual are desirable to reduce the cost and improve accuracy associated with the required sample preparation steps.

### SUMMARY OF THE INVENTION

The present invention relates to a method for determining whether a biological sample was obtained from an individual infected with a virus. Thus, the present method provides a determination of whether the individual donating a particular sample is infected or non-infected. The individual is preferably a mammal, such as a human. The present invention preferably monitors fluorescence of substances associated with the presence of a viral infection in the individual rather than by detecting the virus itself. Thus, the present invention may determine the presence of viral infection even in a sample that contains no actual virus. The present method detects the presence of viral infection without the requirement of directly detecting either the virus itself or virus-directed antibodies.

According to the invention, a sample comprising biological material is obtained from an individual. The sample is preferably a fluid derived from a tissue or a particle-containing fluid derived from a tissue. Plasma is a preferred sample. The biological sample from the individual may be contacted with at least one fractionation reagent, which fractionates the sample into a supemate and a precipitate.

In one embodiment, the fractionation reagent is an organic liquid, preferably comprising at least one member selected from the group comprising alcohols, ketones, nitrites, and aldehydes. In yet another embodiment, the fractionation reagent comprises an organic polymer preferably including at least one of a poly-alcohol, a poly-ether, heparin, dextran sulfate, and combinations thereof. As used herein, the term “organic” refers to carbon containing materials, such as carbon containing polymers.

A fluorescence emission is obtained from a fraction of the sample. The fluorescence emission preferably results from substances that are native to the individual. It should be understood that native substances include substances, such as metabolites, that are only present in an individual when the individual is infected with at least one of HIV and hepatitis. Native substances also include substances that are present in the individual even in the absence of infection but whose abundance changes upon infection. The emission is preferably essentially free from fluorescence resulting from substances that are not-native to the individual. More preferably, the emission is completely free of fluorescence resulting from non-native substances.

Another embodiment of the invention relates to a method for determining whether a biological sample obtained from a mammal is infected with hepatitis. The method comprises obtaining a sample fluorescence emission from the biological sample. At least a portion of the sample fluorescence emission is projected onto a reduced dimension component to determine whether the biological sample is infected with hepatitis. The method may also be used to determine the presence of HIV in a biological sample.

The reduced dimension component is preferably a vector, such as a principle component, obtained by subjecting the control data to a dimension reduction algorithm. Principle components analysis is a preferred dimension reduction algorithm.

### BRIEF DESCRIPTION OF THE FIGURES

The present invention is discussed below in reference to the following figures in which:

FIGS. 1a and 1b illustrate exemplary theoretical fluorescence emissions;

FIGS. 2a and 2b illustrate fluorescence emissions obtained from samples contacted with an acid and acetonitrile;

FIGS. 3a and 3b show fluorescence emissions obtained from samples contacted with isopropyl alcohol;

FIGS. 4a-4d show parameters derived from a subset of the fluorescence emissions of FIGS. 3a and 3b plotted as a function of isopropyl alcohol volume;

FIGS. 5a-5d show parameters derived from a subset of the fluorescence emissions of FIGS. 3a and 3b plotted as a function of plasma volume;

FIGS. 6a and 6b show fluorescence emissions from 19 non-infected samples and from 12 infected samples;

FIGS. 7a and 7b show component factors derived by principle component analysis from the fluorescence emissions of FIGS. 6a and 6b, respectively;

FIG. 8a shows the result of the projection of the fluorescence emissions of FIG. 6a onto the component factors of 7a;

FIG. 8b shows the result of the projection of the fluorescence emissions of FIG. 6b onto the component factors of 7b;

FIGS. 9a and 9b show expanded views of the projections onto factor 4 plotted against the projections onto factor 2 from FIGS. 8a and 8b, respectively;

FIG. 10a shows the average infected and infection-free fluorescence emission from FIG. 6a;

FIG. 10b shows the difference between the fluorescence emissions of FIG. 10a;

FIGS. 11a and 11b show expanded views of the projection of fluorescence emission from another set of samples onto the factors of FIGS. 7a and 7b, respectively;

FIGS. 12a and 12b show expanded views of the projection of fluorescence emission from yet another set of samples onto the factors of FIGS. 7a and 7b, respectively;

FIGS. 13a and 13b show the projection of fluorescence emissions obtained from samples fractionated with a polyethylene glycol onto the factors of FIGS. 7a and 7b, respectively;

FIGS. 14a and 14b show the projection of fluorescence emissions obtained from another group of samples fractionated with PEG onto the factors of FIGS. 7a and 7b, respectively;

FIGS. 15a and 15b show expanded views of the projections of the sample emissions onto factor 4 plotted against the projections onto factor 2 from FIGS. 14a and 14b, respectively;

FIGS. 16a and 16b show projections of emissions from samples fractionated with PEG onto components obtained from fluorescence emissions obtained from ten positive samples and ten negative samples;

FIGS. 17a and 17b show expanded views of the projections onto factor 6 plotted against the projections onto factor 2 from FIGS. 16a and 16b, respectively; and

FIGS. 18a-18d show fluorescence emissions obtained from samples contacted with one of citrate and ethylene diamine tetra-acetic acid (EDTA) anticoagulants.

### DETAILED DESCRIPTION OF THE PREFERRED EMBODIMENT

In accordance with the present invention, a method is provided for determining whether an individual is infected with a virus. In particular, the method is suited for determining whether the individual is infected with at least one of HIV, Hepatitis A, Hepatitis B, and Hepatitis C.

The determination of the presence of infection is based upon the analysis of fluorescence emission from a biological sample obtained from the individual. Infected samples, which are samples obtained from infected individuals, are distinguished from non-infected samples, which are samples obtained from non-infected individuals. Infected samples include, but are not limited to, samples acquired from an individual at any time after the individual has contacted a virus, such as before the individual manifests clinical symptoms associated with the virus. Thus, the present invention is suited for determining the presence of infection during the “window” period when the disease is subclinical, with the individual manifesting no symptoms. Because the present invention does not require the direct detection of viruses, the presence of viral infection in an individual may be determined even though a particular sample obtained from the individual does not contain any viruses.

Biological Samples

The biological specimen or sample used in the present invention may be any biological material such as plasma, serum, blood particles, blood, urine, other bodily fluid, or tissue. Preferably, the specimen or sample is derived from a tissue, such as blood. The term specimen is often taken to mean a biological material that has been subjected to essentially no processing, whereas, the term sample is taken to mean a product that results from even a basic level of processing a specimen. For example, plasma, which is a sample, refers to a fluid that has been derived from a specimen of blood, such as, for example, by treating the blood with anticoagulant and substantially removing cellular material. The method of the present invention, however, may be applied to a substantially unprocessed specimen such as blood or other tissue or to a partially processed sample such as plasma. Thus, the terms specimen and sample are used interchangeably hereinafter. Biological samples suitable for use in the present invention may be obtained from a human, other mammal, or other organism using methods that are known in the art.

Blood specimens are preferably treated with an anticoagulant such as citrate or EDTA to yield plasma. In plasma the preferred amount of citrate is from about 0.013 M, which is typically present in the form of 3.2 mg/ml sodium citrate and 0.42 mg/ml citric acid. Other concentrations can also be used. For example, about 0.0105M citrate (2.47 mg/ml sodium citrate and 0.44 mg/ml citric acid). The amount of citrate is preferably at least about 0.006 M and preferably less than about 0.02 M. The actual amount can vary depending upon the amount of blood collected from an individual.

At least one of dextrose and monobasic sodium phosphate may also be mixed with the plasma. The amount of dextrose, if present, is preferably less than about 10 mg/ml, more preferably from about 3.14 to about 6.3 mg/ml of plasma. The amount of sodium phosphate, if present, is preferably less than about 0.6 mg/ml, more preferably from about 0.1 to about 0.3 mg/ml of plasma.

When K2 EDTA is used as an anticoagulant, the amount of K2 EDTA in the plasma is preferably from about 1 mg/ml to about 3 mg/ml. When K3 EDTA is used as an anticoagulant, the amount of K3 EDTA in the plasma is preferably from about 1 mg/ml to about 3 mg/ml. These values vary again based on the amount of blood actually drawn.

Fractionating Biological Samples

According to the present invention, samples, such as a blood sample mixed with at least one of EDTA, dextrose, and monobasic sodium phosphate, may be contacted with at least one reagent to enhance the ability to distinguish non-infected samples from infected samples. The reagent is preferably a fractionation reagent contacted with the sample under conditions suitable to fractionate the sample into first and second fractions.

In one embodiment of the present invention, the reagent is an organic fluid, such as an organic solvent. The organic fluid preferably fractionates the sample into a first fraction enriched in moderately hydrophobic fluorophores, such as lipoproteins and other small organic molecules native to the organism from which the sample was obtained. In the lipoprotein enriched fraction, the amounts of proteins other than lipoproteins are relatively reduced compared to the relative amounts of the lipoproteins and proteins present in the sample prior to fractionation.

The first fraction is preferably a supemate, which can be separated from the second fraction, which is preferably a precipitate, by traditional methods such as filtration or centrifugation. In general, the second fraction comprises hydrophilic molecules such as proteins, which precipitate from the sample. The preferred method employs final concentrations of from about 30% to 70% organic liquid and about 10% to 60% (v/v) plasma.

A preferred organic liquid is an alcohol, such as an aliphatic alcohol having less than about 11 carbon atoms. Three carbon alcohols such as isopropyl alcohol are preferred. Other suitable organic liquids include, nitrites, such as acetonitrile, aldehydes, such as acetaldehyde, ketones, such as acetone, and hydrocarbons, such as hexane. Organic solvents such as carbon disulphide may be used. Mixtures or derivatives of any of the above-mentioned organic liquids may also be used. For example, molecules containing one or more alkene groups, halogen atoms, or aromatic groups are suitable for use with the present invention. In addition to the organic liquid, other substances may also be contacted with the sample. These substances include, for example, water, and salts or buffers, such as ethylene diamine tetra-acetic acid (EDTA) or citrate salts.

A sample may be directly contacted with the organic liquid upon obtaining the sample. For example, the organic liquid and other substances, if any, may be placed in a specimen vial into which an individual's blood is drawn. Once collected, the mixture of the sample and organic liquid may be analyzed immediately, as discussed below. Alternatively, the mixture may be sent to an analysis site, which receives and analyzes samples obtained from individuals at a number of different sites.

A second embodiment of the present invention also includes fractionation of the sample into at least a first fraction that is enriched in at least some lipoproteins and a second fraction having a reduced amount of those lipoproteins. The fractionation includes forming a mixture by contacting the sample with a reagent that fractionates the sample into a supernate and a lipoprotein enriched precipitate. The mixture may also include other fluids, such as water or organic solvents, and salts or buffers, such as ethylene diamine tetra-acetic acid (EDTA) or citrate salts.

The reagent that precipitates a lipoprotein enriched fraction is preferably a polymeric substance, such as a poly-ether or derivative thereof. Preferred poly-ethers include polyethylene glycol (PEG) or a combination of polyethylene glycols. Polyethylene glycols have the general formula H(OCH2-CH2)nOH, where n is an integer greater than 3. The name of each PEG includes a numerical designation that is related to its molecular weight. For example, a preferred poly-ether, PEG-6000, has an average molecular weight of between about 5000 and 7000 grams per mole. Other suitable PEG's include, for example, PEG-200, PEG-400, PEG-600, PEG-900, PEG-1000, PEG-1500, PEG-4000, PEG-6000, PEG-8000, or combinations thereof.

Alternatively to PEG's, other polymeric substances including poly-anionic substances, such as heparin, dextran sulfate, and phosphotungstic acid, may be used singularly or in combination to obtain a lipoprotein enriched fraction. One or more of these alternative poly-anionic substances may also be combined with one or more PEGs.

At room temperature (20° C.) many polymeric substances may behave like viscous waxes, which would inhibit fractionation. Therefore, the sample and polymeric substance are preferably combined under conditions in which the combined polymeric substance and sample possesses sufficient fluid-like properties to facilitate the formation of a fraction enriched in lipoproteins and a fraction having a reduced amount of those proteins. Fluid-like properties include, for example, a low enough viscosity to permit flow. The sample is preferably combined with a mixture of the polymeric substance and a fluid, such as water or a buffer solution. The mixture of the polymeric substance and fluid exhibits sufficient fluid-like properties to facilitate fractionation upon combination with the sample. In a preferred embodiment, the sample is combined with a mixture comprising from about 20% to about 90% polymeric substance and about 80% to about 10% aqueous buffer solution. More preferably, the mixture comprises from about 40% to about 70% polymeric substance and about 60% to about %30 aqueous buffer solution. The buffer solution is preferably a phosphate buffer solution, as described above.

Alternatively, or in combination with forming a mixture of the polymeric substance and buffer, the sample may be combined with a higher concentration of polymeric substance at a temperature sufficient to impart the fluid-like properties to the combined polymeric substance and sample. Many poly-ethers, for example, have melting points of less than about 100° C. and are suitable for use in the present method.

While poly-ether soluble substances are extracted from the sample into the poly-ether, poly-ether insoluble substances preferably separate from the poly-ether. Separative techniques such as for example centrifugation or filtration of the solution may be used to maximize the recovery of poly-ether insoluble substances, which are recovered as a solid or semi-solid mass such as a pellet. The recovered insoluble substances are typically rich in lipoproteins because these substances tend to be insoluble in the poly-ether. Prior to fluorescence analysis, as discussed below, the lipoproteins in the recovered mass may be suspended in a fluid, such as a water buffer mixture or an organic liquid such as one of the above-mentioned organic liquids.

In a third embodiment of the invention, an acid, preferably an organic acid comprising at least one halogen, such as fluorine, is contacted with the sample. In this regard, a spectrophotometric grade of acid is well suited for use in the present invention. For example, spectrophotometric grade trifluoroacetic acid has minimal absorbance at ultraviolet and visible wavelengths greater than about 270 nm and has minimal fluorescent impurities. Generally, the sample and acid may be combined by mixing, although homogenization or digestion may also be desirable in order to achieve a substantially homogeneous mixture.

In addition to acid, the sample is preferably contacted with a buffer to maintain the pH of the sample within an appropriate range for dissociating proteins from compounds that are carried by or otherwise associated with the proteins, as discussed below. For example, pterins are dissociated from proteins at a pH of about 4. Preferably, the pH of the buffer is between about 2.5 and 5.5, such as, for example, between about 3.5 and 4.5, and most preferably about 4. The buffer and sample are preferably combined prior to adding the acid but the order of combination may be reversed or performed simultaneously.

One of ordinary skill in the art understands how to prepare a buffer according to the desired pH and buffer capacity relative to the amount of plasma or other biological sample used. Preferably, the buffer solution comprises from about 0.6 to 1.3 molar buffer, for example, between about 0.9 and 1.1 molar and most preferably around 1 molar buffer. To avoid overly diluting the biological sample and compounds therein, the volume of buffer solution is preferably from about 10 to 50% as large as the volume of biological sample, preferably from about 15 to 25% as large. The exact volume and buffer concentration, however, may be determined by one of ordinary skill in the art and depends, for example, on the particular biological sample chosen for analysis, the diseased or infectious state in question, and the amount of acid used, as described below.

The mixture of sample and acid preferably comprises more than about 60% by volume acid, for example, more than about 90%, or most preferably, more than about 99% acid by volume. When the biological sample is plasma, the volume of organic acid added to the first mixture is preferably from about 20% to 75% as large as the volume of buffer used, preferably from about 40 to 60% as large.

Biological samples obtained from mammals comprise a number of lipophilic compounds, such as pterins, that are associated or otherwise bound with proteins. The amount of acid combined with the sample is preferably sufficient to separate at least some of these protein-associated compounds from their associated proteins. The exact amount and strength of acid, however, may be determined by one of ordinary skill in the art and depends, for example, on the buffer used, the particular biological sample chosen for analysis and the diseased or infectious state in question.

The sample contacted with the acid is fractionated to obtain a first fraction enriched in the molecules dissociated from the protein molecules in the sample. A second fraction is enriched in hydrophilic proteins leaving the first fraction depleted in these proteins. To fractionate the sample, an organic liquid is combined with the acid buffer sample mixture. Any of the organic liquids discussed above that fractionate the mixture into a first fraction comprising detectable amounts or concentrations of the dissociated compounds may be used. Acetonitrile is a preferred liquid. Spectrophotometric grades of acetonitrile, which have minimal fluorescent and absorbing impurities, are most preferred.

The volume of the organic liquid should not be so large as to overly dilute compounds extracted from the biological sample. For example, when the biological sample is plasma, the volume of organic liquid, such as, for example, acetonitrile, is preferably about 5 to 25 times as large as the volume of plasma, more preferably about 12 to 18 times as large. The exact amount of liquid, however, may be determined by one of ordinary skill in the art and depends, for example, on the particular biological sample chosen for analysis and the diseased or infectious state in question.

To assist in the formation of a fractionated sample, the mixture may be heated either before or after the organic liquid is added. Preferably the mixture is heated after adding the organic liquid. The mixture should be heated to a temperature and for a period of time sufficient to dissociate compounds from the proteins or other biological molecules in biological sample. For example, the mixture may be heated to from about 65 to 115° C., preferably to from about 90 to 110° C. Preferably, the mixture is heated for more than about one-half hour, preferably for more than about 1 hour. Following such a heating step, a first fraction comprising a substantially clear supernate is formed over a second fraction comprising solids, which are partially or wholly insoluble in the supernate.

At least in part, the solids comprise precipitated proteins. Compounds, previously associated with the proteins or other biological molecules within the biological sample are substantially dissociated from the proteins and extracted into the supemate. The supernate and solids may be separated using techniques known in the art, such as filtration, centrifugation, selective adsorption, or combination thereof.

Spectroscopic Analysis

Once the sample has been prepared according to one of the fractionating methods discussed above, the sample is subjected to spectroscopic analysis. Particularly powerful approaches for analyzing such samples is described in U.S. patent application Ser. No. 09/224,141 filed Dec. 31, 1998 and Ser. No. 09/436,207 filed Nov. 8, 1999, which are incorporated herein by reference in their entireties. Briefly, these applications describe instrumentation and algorithms suitable for discriminating a diseased or infected sample from a disease-free or infection free sample.

In general, the spectroscopic analysis involves obtaining at least one fluorescence emission that results from one or more substances that are native to the individual from which the sample was obtained. Native substances include, for example, biological substances produced by the individual, metabolites of substances produced by the organism, and metabolites of substances, such as food and pharmaceuticals, ingested by the individual. Substances that are only present in samples drawn from an infected individual are considered to be native substances.

Although the present methods may modify at least one of the environment, conformation, and structure of native substances, the modified substances are nonetheless considered to be native substances. For example, as discussed above, native substances associated with proteins may become dissociated from the proteins during fractionation of the sample. The dissociated proteins and substances are native because they originated from the individual providing the sample. On the other hand, non-native substances include, for example, radiological tags, fluorescent tags and fluorescent stains. In particular, a non-native substance is a substance added to the sample with the objective of measuring a spectroscopic or radiological signal resulting from the non-native substance. The samples of the present invention are preferably essentially free of non-native fluorescent substances, such as radiological tags, fluorescent tags, and fluorescent stains.

According to the invention fluorescence emission obtained from a sample is preferably essentially free of fluorescence emission resulting from substances non-native to the organism from which the sample was obtained. More preferably, the fluorescence emission is completely free of fluorescence emission resulting from non-native substances. By essentially free, we mean that the fluorescence emission does not include fluorescence emission resulting from non-native substances in an amount that is sufficient to inhibit the determination of the infection status of the organism based upon the fluorescence emission resulting from one or more native substances. For example, the essentially free fluorescence emission includes an insufficient amount of non-native fluorescence emission to significantly reduce the accuracy and/or precision of a determination of an organism's infection status compared to a determination based upon fluorescence emission that is completely free of emission resulting from non-native substances. Preferably, the fluorescence emission includes a contribution of less than about 10%, preferably less than about 5%, and most preferably less than about 2.5% emission from non-native substances.

Acquisition of a fluorescence sample from the presently prepared samples comprises irradiating the sample with radiation having a sufficient energy for inducing a fluorescence emission from native substances in the sample. The sample is irradiated with at least one wavelength of at least 190 nm, preferably at least 270 nm, and most preferably at least 310 nm. The wavelength is preferably less than about 750 nm such as less than 400 nm.

The preferred device for obtaining fluorescence emission employs a laser as excitation source. Lasers such as, for example, a Nd:YAG laser that emits a fundamental or harmonic line in the ultraviolet are ideal excitation sources. A preferred laser is a diode-pumped frequency-tripled NdYAG, with average power output of approximately 2 mW at 355 nm. in a beam of diameter approximately 0.5 mm FWHM. A filtered non-laser source, such as a mercury lamp, a halogen lamp, or an arc lamp, may also be used.

The laser beam impinges upon the sample, which is preferably a fluid contained in a flow cell formed of suitable optical material such as quartz. A typical flow cell is rectangular in shape with mirrored faces to improve excitation and collection efficiency. When the sample is irradiated with light, such as the laser beam, fluorescence emission results from fluorescent substances present in the sample. The fluorescence emission preferably impinges upon a long-pass filter configured to remove light at the excitation wavelength from the light that impinges upon the detector.

The fluorescence is collected by a short focal-length, low f-number quartz lens and focused into a first end of a 1 mm diameter fiber bundle. Light exits a second end of the fiber and is introduced to a grating spectrometer. Fluorescence is preferably collimated onto a grating generating a spectrum by dispersing the light as a function of wavelength. According to the present method fluorescence spectrum comprising a plurality of fluorescence intensities each obtained at a respective one of a plurality of different wavelengths is a preferred fluorescence emission. Alternative dispersive optical elements such as prisms may be used to obtain a fluorescence spectra from the fluorescence emission. Additionally, suitable fluorescence spectra may be obtained using interferometry or a plurality of different optical filters, each filter passing a different range of wavelengths.

The spectrum is focused onto a cooled CCD array, whose analog electronic output is converted to a digital representation of the spectrum and transferred to a digital computer in which an analysis algorithm, discussed below, is applied to the fluorescence emission. Fluorescence spectra are typically represented as intensity-wavelength data or intensity-frequency data where the intensity is given as a function of the wavelength or frequency of the fluorescent light, respectively.

In terms of wavelength, the spectrum of the detected fluorescence emission comprises at least one intensity maximum at a wavelength of from about 400 to 850 nm, more preferably at from about 400 to 600 nn. Preferably the intensity at wavelength of maximum intensity is at least about 20% greater than a non-maximum intensity at a second, different wavelength in the range between about 400 and 850 nm.

Fluorescence Emission Analysis

Once a fluorescence emission is obtained from a sample, one or more parameters such as, for example, the total fluorescence, the wavelength of peak intensity, the shift in the wavelength of peak intensity, the peak intensity, or fluorescence lifetime may derived from the fluorescence emission. The one or more parameters may be used to discriminate samples obtained from infected and non-infected individuals. Preferably, the sample emission parameter is compared to a control parameter obtained from at least one control sample having a known infection status. Alternatively, the control parameter may be obtained from a plurality of fluorescence emissions, each emission resulting from a sample obtained from a respective individual having a known infection status (either infected or non-infected). Several parameters are discussed below by way of example only.

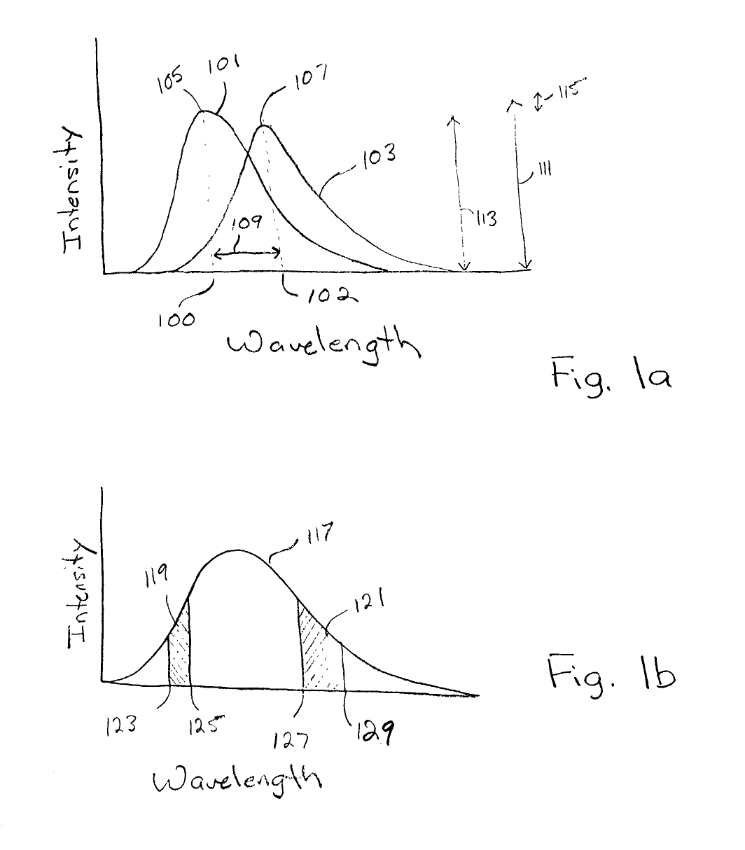

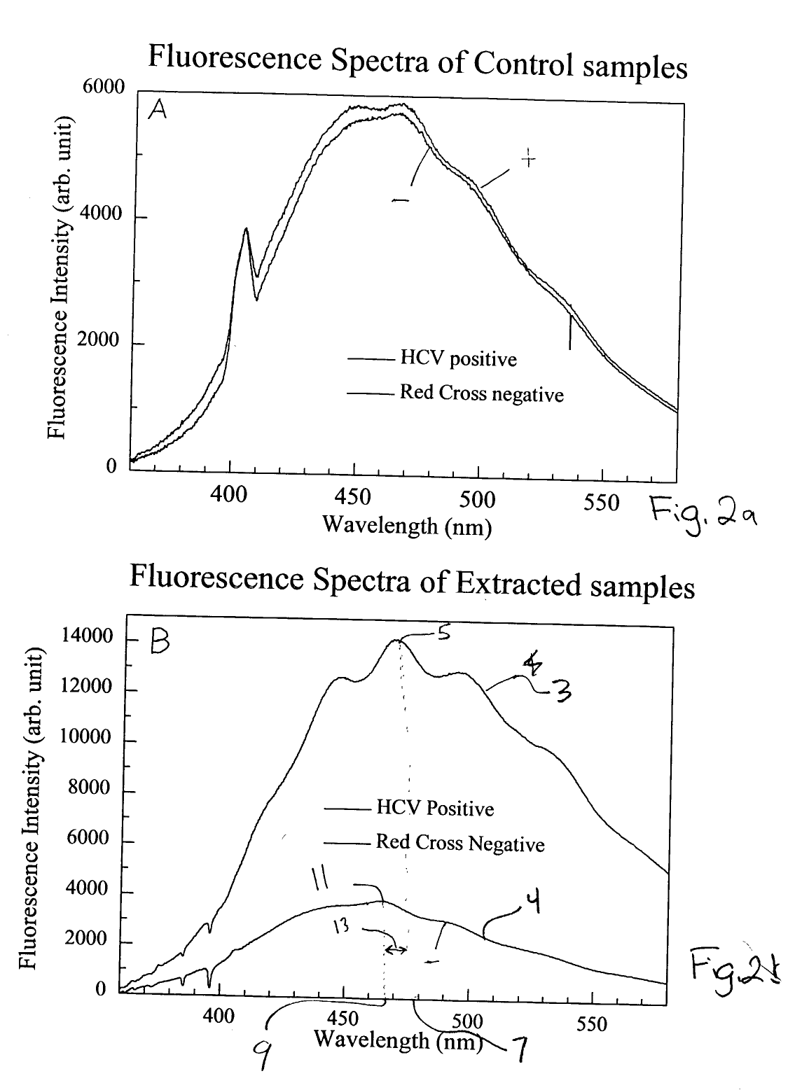

One parameter is the wavelength or frequency at which the maximum of fluorescence intensity occurs (λp). Mathematically, this is when dY/dX=0, where Y is the intensity of fluorescence and X is wavelength of the fluorescence. Referring to FIG. 1a, which illustrates a fluorescence emission by way of example, a first emission 101 exhibits a fluorescence intensity maximum 107 at a wavelength 100. A second emission 103 exhibits a corresponding fluorescence intensity maximum 107 at a wavelength 102.

Another parameter is the amplitude of the peak fluorescence at a predetermined wavelength or frequency of the fluorescence emission (Am). For example, FIG. 1a shows that a peak amplitude 111 of emission 101 is different by an amount 115 from a peak amplitude 113 of fluorescence emission 103.

A third parameter is an area ratio (Ar). Referring to FIG. 1b, the intensity of a fluorescence emission 117 is shown as a function of wavelength. First and second areas 119 and 121 are derived from emission 117. Area 119 is defined as the integrated area beneath fluorescence emission 117 taken from a first wavelength 123 to a second wavelength 125. Similarly, area 121 is defined as the integrated area beneath fluorescence emission 117 taken from a first wavelength 127 to a second wavelength 129. An area ratio is derived by the forming the ratio of the first and second areas. When the first and second wavelengths of each area are identical, the area ratio parameter collapses into a ratio of intensities.

A parameter derived from a fluorescence emission resulting from a biological sample obtained from an individual having an unknown infection status may be compared with a second parameter to determine whether the individual is infected or infection free. Referring to FIG. 1a, for example, a comparison of wavelength 100 and wavelength 102 is illustrated. In one embodiment of the invention, a comparison is performed by deriving a difference 109 between wavelengths 100 and 102. The difference between two parameters is indicative of the presence or absence of infection in the individual having unknown infection status. The comparison is preferably a determination of whether a first parameter, which is derived from emission obtained from the sample having unknown infection status, is greater or less than the second parameter, which is preferably derived from at least one sample having a known infection status. Preferably, difference 109 is compared to a threshold value to determine the presence of infection in the biological sample from the individual of unknown status. When a difference between an emission parameter from a first sample and a control emission parameter derived from a non-infected sample exceeds the threshold value, the presence of infection in the first sample is indicated.

A threshold value is a parameter preferably derived from set of fluorescence emissions comprising a plurality of member fluorescence emissions. Each of the member emission results from a respective control sample obtained from an individual having a known infection status. Control samples may be obtained from both infected and infection free individuals. Upon obtaining the set of fluorescence emissions, at least one parameter, such as, for example, a wavelength of maximum intensity, is derived from each member emission. Because the set comprises member emissions obtained from both infected and infection free individuals, characteristic values of parameters of the emissions may be determined. These characteristic parameters may be used to derive a threshold value, which is parameter that discriminates infected and infection free samples.

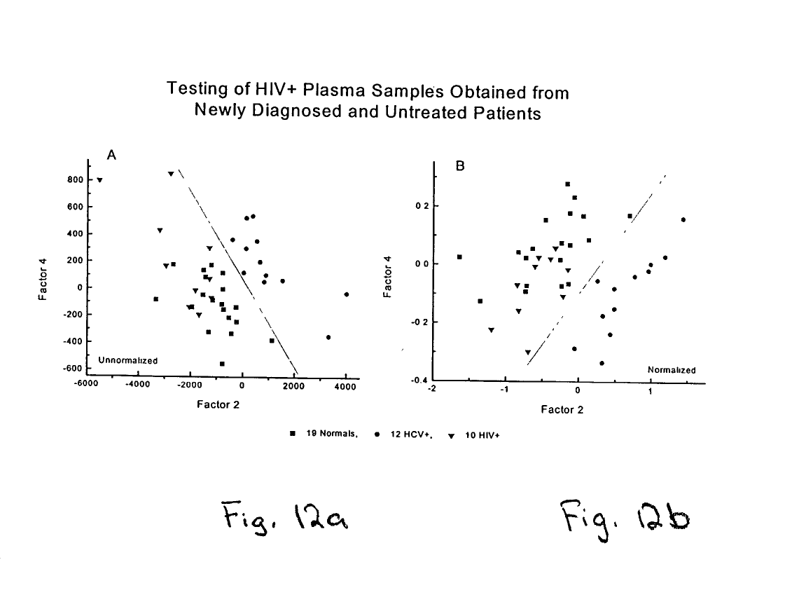

One method for determining a threshold value is to determine the average value of a parameter derived from emissions obtained from either infected or non-infected samples. For example, the average difference between the wavelengths of maximum intensity of emission from infected and non-infected samples may be determined from a plurality of control samples. Based on the average difference and the uncertainty of the measurements, a threshold value parameter may be derived. Once a fluorescence emission is obtained from a sample having an unknown infection status, a parameter corresponding to the threshold parameter is derived from the fluorescence emission. In this example, the wavelength of maximum intensity parameter is derived from the fluorescence emission resulting from the unknown sample. A second difference is then determined between the wavelength of maximum intensity parameter of the unknown sample emission and a corresponding parameter derived from at least one control emission. The second difference is then compared to the threshold value parameter to determine whether the unknown sample was obtained from an infected individual. Differences between emission derived from infected samples and non-infected samples are discussed below in reference to FIGS. 10a and 10b.

In accordance with one embodiment the present invention, one or more of these parameters are used to discriminate infected samples from non-infected samples. For example, peak wavelength may be used to discriminate infected samples, such as HIV positive samples, from uninfected samples, such as HIV negative samples.

In accordance with the present invention, various discriminators are provided for discriminating infected samples from non-infected samples. In a preferred embodiment, a discriminator D1 is constructed from 1/λₚ, Am and Ar, all three of which will be greater for positive samples than negative samples or the mean value from a normal data base. Thus, a threshold parameter derived from any additive or multiplicative combination of these parameters from positive samples divided by an identical combination of these parameters from the normal data base will be greater than 1. The value will be closer to 1 or less than 1 for negative samples. Such an analytic function for D1 is expressed as D1=f(1/λₚ, Am, Ar).

Another discriminator D2 takes advantage of the differential shifts in one or more of these parameters (1/λₚ, Am, Ar) after the sample undergoes treatment by one of the fractionation methods of the invention. After fractionation, Δλₚ, is greater for HIV positive samples than for HIV negative (normal) samples. Because ΔAm is greater for HIV negative samples than for HIV positive samples, the reciprocal of the difference in Am, 1/ΔAm, is greater for positive samples. The parameter ΔAr is greater for positive samples. Thus, any additive or multiplicative combination of these parameters from positive samples divided by an identical combination of these parameters from the normal data base will again be greater than 1. This ratio for negative samples will be closer 1 or less than 1. Such an analytic function for D2 is expressed as D2=f(Δλₚ, Δ1/Am, Aar). Thus, in one embodiment of the invention, a parameter from a spectrum derived from an untreated sample is compared with a parameter derived from a treated sample. By treated it is meant that the sample has been subjected to a fractionation treatment as discussed above.

In accordance with the present invention, an aggregate discriminator D*=f(iD1, jD2, . . . , kD3) may be provided, which will yield values for HIV positive samples greater than for HIV negative samples. The coefficients i through k are weighing factors to be determined empirically. HIV negative samples will yield a range of D*s, the uncertainty of which within the data base for the normal samples (i.e., HUV negative samples) must be determined.

Referring to Fig. *, one embodiment includes use of a parameter obtained by reducing a dimensionality of

One embodiment for determining whether a biological sample obtained from a mammal is infected with hepatitis or HIV includes using a dimension reduction algorithm, such as principle components analysis, factor analysis, or singular devalue decomposition, to obtain a reduced dimension data set from a set of control data comprising a plurality of fluorescence emissions from respective control samples having a known infection state. The reduced dimension data set comprises one or more reduced dimension components, which are preferably vectors such as principle components.

A sample fluorescence emission is obtained from biological sample using, for example, any of the techniques described herein. The sample fluorescence emission may be essentially free of fluorescence resulting from substances not-native to the mammal.

The sample fluorescence emission and the reduced dimension component are projected onto one another to determine whether the biological sample is infected with hepatitis.

### EXAMPLES

The present invention is further described in the following non-limiting examples.

### Example 1

Referring to FIG. 2a, a sample of plasma from a Hepatitis C infected individual and a control sample from an individual free of Hepatitis C were obtained. A fluorescence instrument comprising an ultra-violet excitation source and a multiwavelength detector was used to obtain a Hepatitis C fluorescence emission spectrum 1 and a control fluorescence emission spectrum 2 from the Hepatitis C infected sample and Hepatitis C free control sample, respectively. Spectra 1,2 were obtained prior to treatment with the method of the present invention. Although Hepatitis C spectrum 1 exhibits a somewhat larger intensity than control spectrum 2, the spectra are not substantially resolved and it is difficult to discriminate between the infected and non-infected samples on the basis of these spectra.

Subsequently, a portion of each plasma sample was treated separately according to the following steps:

A) A 0.5 ml volume of each plasma sample was combined with a 0.1 ml volume of 1 M phosphate buffer of pH 4, a 0.05 ml volume of trifluoroacetic acid, and a 7.5 ml volume of acetonitrile.

B) The mixture was heated at 100° C. for two hours to obtain a second mixture comprising a clear supemate and partially or wholly insoluble solids.

C) The clear supemate was separated from the solids.

Subsequently, the clear supernatant obtained from the Hepatitis C plasma and the control plasma were subjected to fluorescence analysis as described for the untreated samples above to obtain a treated Hepatitis C spectrum 3 and a treated control spectrum 4, respectively, as shown in FIG. 2b.

A first parameter of the hepatitis C spectrum 3 may be compared to a parameter of the control spectrum 4 to determine that the hepatitis spectrum 3 resulted from a sample obtained from an infected individual. For example, a parameter of hepatitis spectrum 3 is a wavelength 7 of an intensity maximum 5. A second parameter of control spectrum 4, is a wavelength 9 of an intensity maximum 11. A comparison between the parameters may be performed by determining a difference 13 between wavelengths 7 and 9. In performing such a comparison, it may be determined that a sample was obtained from an infected individual when the difference between parameters exceeds a threshold value, which may be calculated as discussed above. Like wavelength 7, difference 13 is a parameter of fluorescence emission 3.

Comparisons of other parameters of the spectra are also indicative of the presence of infection in the individual donating the hepatitis c sample. For example, a peak amplitude and area of treated Hepatitis C spectrum 3 are both significantly greater than a peak amplitude and area of treated control spectrum 4. Moreover, the treated and untreated spectra exhibit a different wavelength of maximum intensity. For example, the hepatitis C spectrum exhibits a red shift (shift to longer wavelengths) compared to the treated control spectrum. These distinctions between the treated spectra are significantly greater than the differences between the spectra of the untreated plasma samples. This indicates that the treating the samples according to the present invention provides the ability to more selectively distinguish between positive and negative samples. The results clearly demonstrate that the method of the present invention dramatically increases the differences between fluorescence emission obtained from diseased or infected samples and disease-free or infection free samples.

### Example 2

Recently drawn plasma samples were obtained from newly diagnosed HCV patients who had not as yet received treatment and from uninfected control subjects. These patients were also screened for non-hepatitis liver disease. Normal samples were received concurrently for comparison. For the examples discussed here plasma prepared with citrate were used for isopropyl alcohol studies unless otherwise specified, while samples subjected to PEG fractionation utilized plasma prepared in EDTA.

Samples were also obtained from treatment naïve HIV-infected patients by methods identical to those employed for the HCV patients noted above. Additional HCV positive plasma samples were obtained from a commercial blood bank. These blood bank samples were taken from plasma units that had been derived from processing of the whole blood donation and represent a higher level of dilution (20-30%) than the newly samples from the newly diagnosed patients.

Isopropyl Alcohol Fractionation

A first set of samples were prepared using isopropyl alcohol fractionation. Sample amounts of 0.5 ml plasma were combined with 0.75 ml DI H2O and 1.25 ml 100% (v/v) isopropyl alcohol (HPLC grade) to obtain a 2.5 ml total volume. The reagents were stored, and assay was conducted, at about 20° C. The components were mixed for 2-3 seconds by vortex and incubate at about 20° C. for 5-10 minutes. Then, the mixture was centrifuged for 5 minutes at 3150 RPM using a Sorvall RT 6000B centrifuge. Following centrifugation, the supernatant was carefully removed from the pellet and spectroscopically analyzed with a 355 nm ND:Yag laser based spectrofluorometer (LBS) or OPO system.

Polyethylene Glycol Fractionation

A second set of samples were analyzed using fractionation with polyethylene Glycol (PEG). This fractionation procedure was performed on 1.0 ml of plasma prepared in EDTA, as discussed above. Plasma was mixed with 0.2 ml of a 60% solution of PEG-6000 and PEG insoluble material allowed to precipitate at 20° C. for 15 minutes. The insoluble material was obtained by centrifugation. The insoluble material was suspended to a total volume of 240 ml with NaCl containing EDTA and a 90 ul aliquot diluted to 4.0 ml for analysis by fluorescence spectroscopy, as described above.

Fluorescence Emission

In the present example, the fluorescence emission spectra of samples prepared by both methods were obtained at several excitation wavelengths as listed below. For most studies and the standard method, 355 nm excitation light was generated with a Uniphase frequency tripled YAG laser and fluorescence emission was collected with fiber optics and channeled to a spectrometer/CCD system in the Seroptix ID-LBS device. In this system spectral data was collected during sample flow through a 16 ml flow cell.

All other excitation wavelengths were generated with a Spectra Physics OPO tunable laser system and fluorescence emission was collected in manner similar to that with the ID-LBS except that standard fluorescence cuvettes were used under non-flow conditions. Excitation wavelengths of 290, 320, 355, and 380 nm were used.

Data Analysis Raw emission data from the CCD were processed to provide a preliminary assessment of emission parameters including wavelength of maximum intensity, peak amplitude, average intensity, and peak area ratio. In the present example, the first area was the integrated area from 412 nm to 460 nm and the second area was the integrated area from 550 to 550 nm.

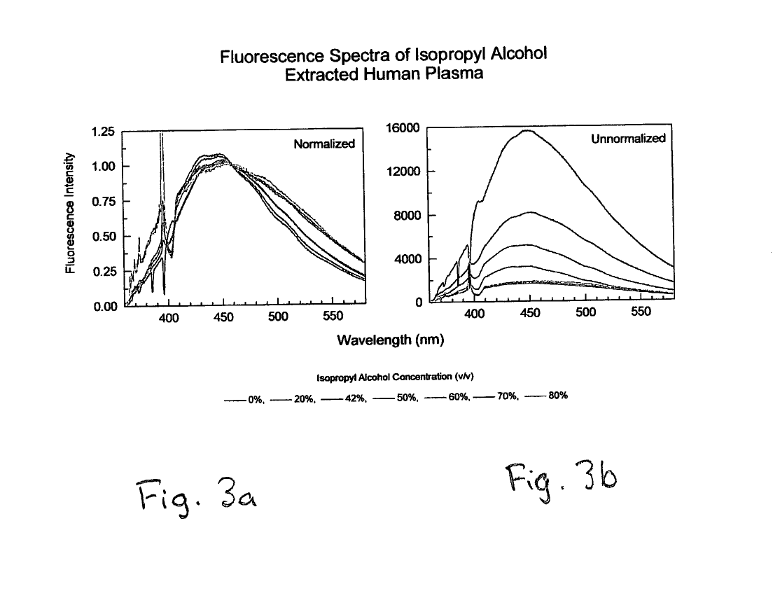

Referring to FIGS. 3a and 3b, spectra are typically displayed as both laser-power normalized spectra and point normalized spectra. The latter are referred to as the normalized spectra. In the original spectral data space, red lines represent spectra from positive plasma samples and black lines are spectra from normal samples. Note that the Raman spectrum of water has been subtracted from these fluorescence spectra, however, a Raman peak of isopropyl alcohol is prominent at around 400 nm.

Results

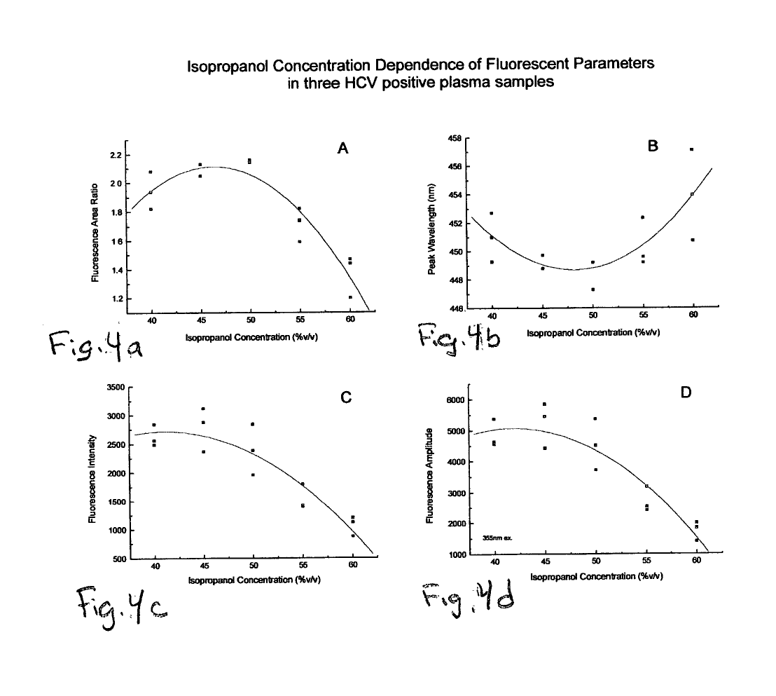

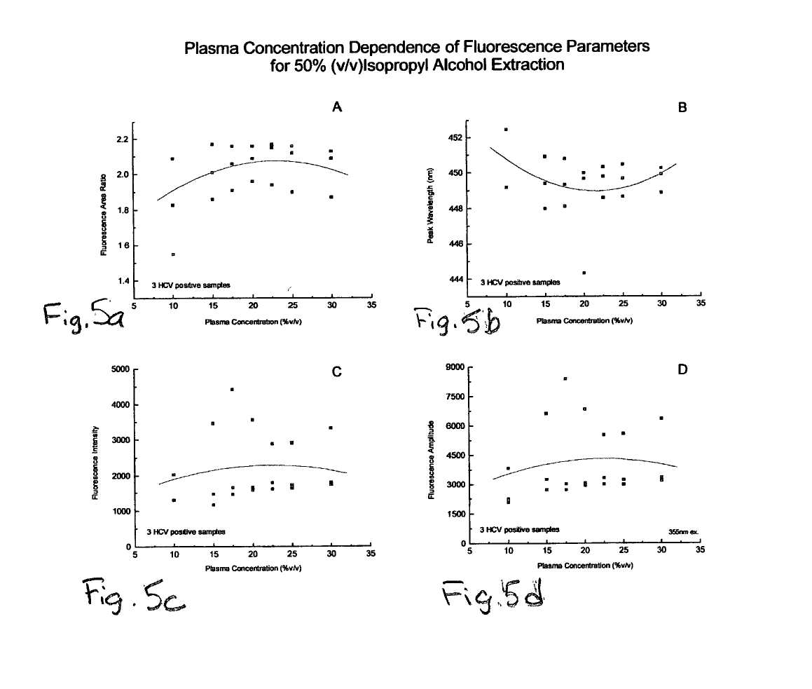

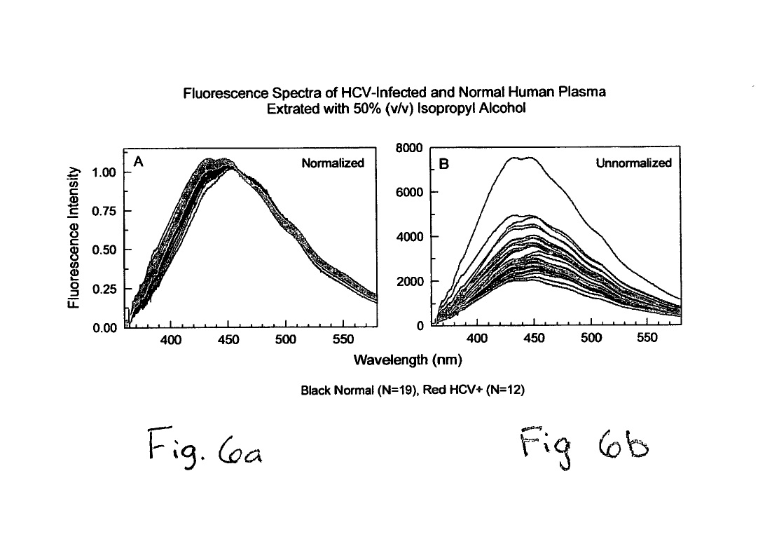

Recall that differences between parameters obtained from infected and non-infected samples are indicative of the presence or absence of disease. Thus, the method optimization seeks to determine IPA concentrations that maximize differences between the parameters. Of all samples tested from 0 to 80% IPA, the sample treated with 50% IPA presented the most significant low wavelength peak in normalized spectra suggesting selective extraction of a minor species under these conditions. For confirmation of the IPA concentration optimization, three HCV positive plasma samples were fractionated in triplicate at concentrations of IPA ranging from 40 to 60% at a constant volume and a plasma concentration of 20% (v/v). Analysis of fluorescence generated from 355 nm laser excitation revealed subtle differences in emission parameters. For example, FIG. 4b shows that 50% IPA (v/v) provided the largest shift to lower wavelengths for the wavelength of maximum intensity. An IPA concentration of about 50% also produced the highest fluorescence area ratio between infected and non infected samples, as seen in FIG. 4a. The larger shifts improve the ability to discriminate infected from non infected samples.

50% IPA produced marginally lower fluorescence intensity (averaged fluorescence over full spectral range) and amplitude (fluorescence signal at peak wavelength) (FIGS. 4c, 4d) than lower IPA concentrations, however, for method optimization these parameters were considered secondary since correction of these factors by normalization would be possible.

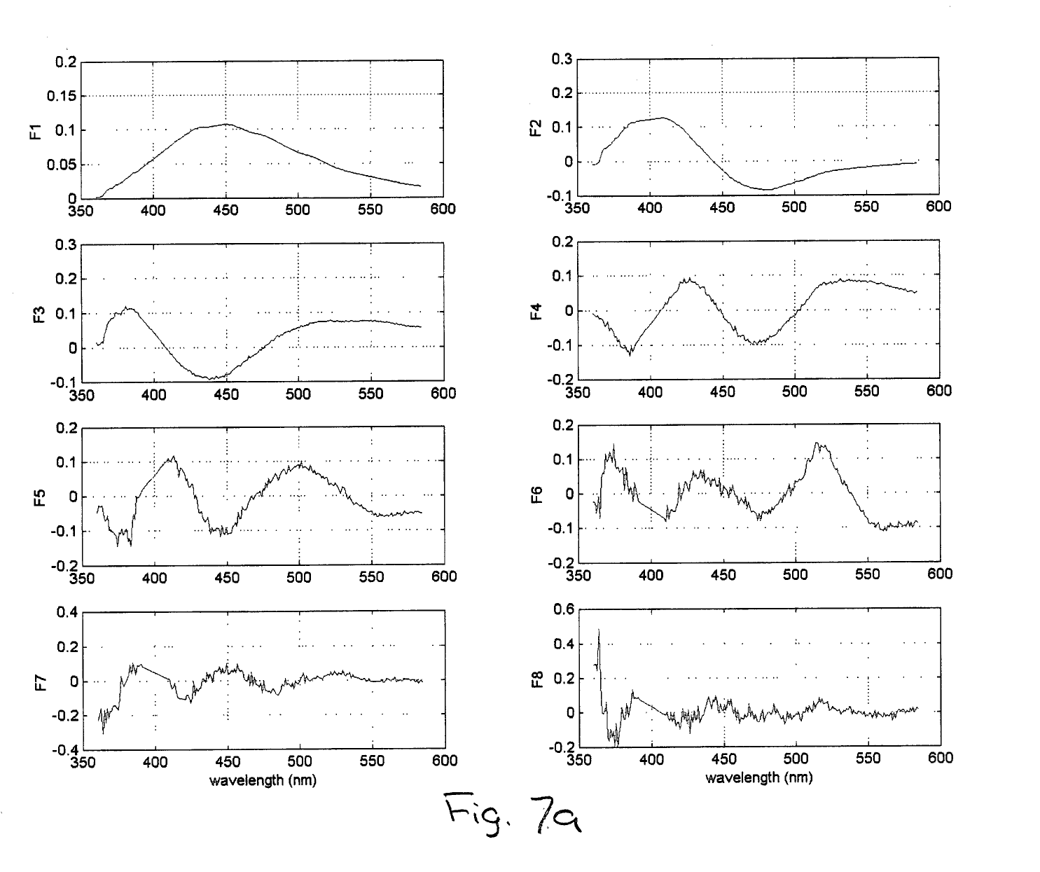

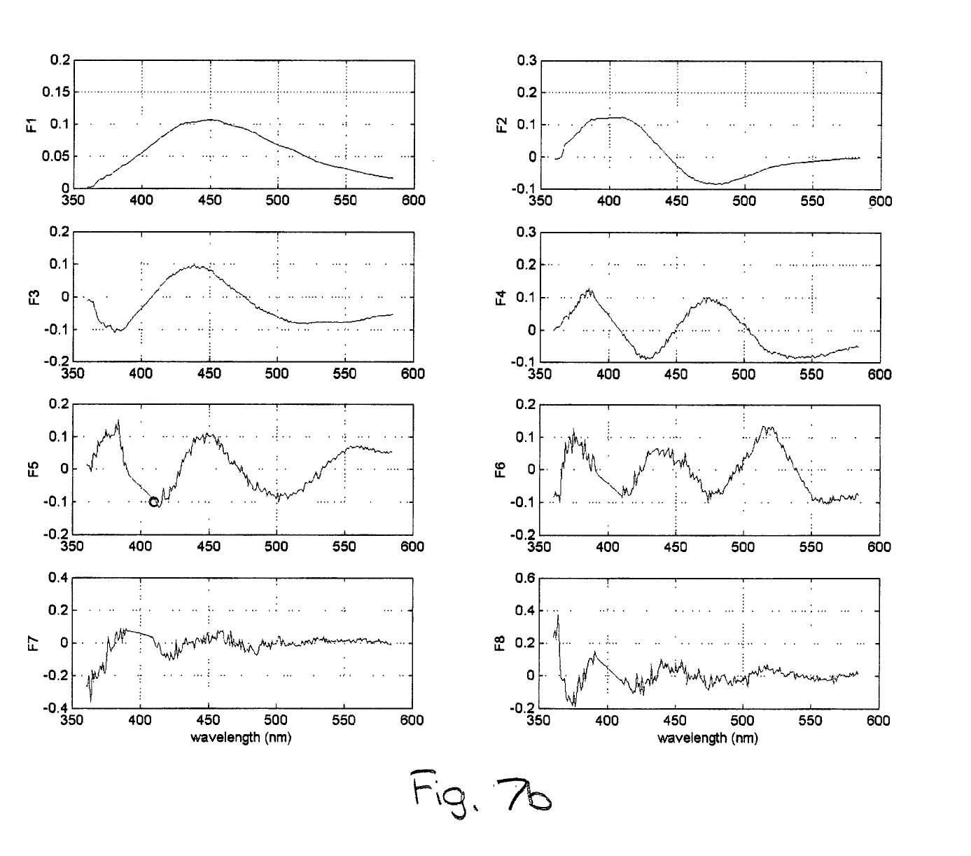

Referring to FIGS. 5a-5d, the area ratio, peak wavelength, intensity, and amplitude were evaluated from fluorescence emission obtained as a function of plasma concentration at constant 50% (v/v) IPA. There is an approximate linear relationship between fluorescence peak amplitude or intensity and plasma concentration in the range from 5 to 20% (v/v) final concentration. At 25% (v/v), little increase was seen in these parameters suggesting that the extract was approaching saturation above 20% (v/v) plasma. A more detailed assessment of the optimal plasma concentration was conducted, varying the final amount of plasma from 10 to 30% (v/v) at constant volume and 50% (v/v) isopropyl alcohol. Samples obtained from three HCV-positive blood donors were assayed at each concentration in triplicate. Mean values for each donor were graphed as individual points to represent concentration dependent variation in quantitative spectral parameters. For each parameter, the data were fit with a binomial curve as an alternative assessment of optimal levels.

Over the plasma concentration range tested, peak area ratio was maximal (FIG. 5a) and peak wavelength was minimal (FIG. 5b) at approximately 20% (v/v) plasma. Fluorescence intensity (FIG. 5c) and amplitude (FIG. 5d) showed approximately linear increases over this plasma concentration range with the determination of an optimum obscured by the high level of donor variability regarding these parameters. For each donor sample, adequate intensity for spectral deconvolution approaches was obtained in the 20% (v/v) sample which, based on the peak wavelength and area ratio parameters, was selected as the tentative optimal concentration for further comparative testing of HCV positive and negative plasma samples.

Additional optimization of the time of incubation between addition of isopropyl alcohol and initiation of centrifugation, and time of centrifugation, showed little difference between 2 and 20, and 5 and 20 minutes respectively. A 10 minute incubation and 5 minute centrifugation time were adopted as standard conditions. All studies were performed at room temperature, i.e., about 20° C.

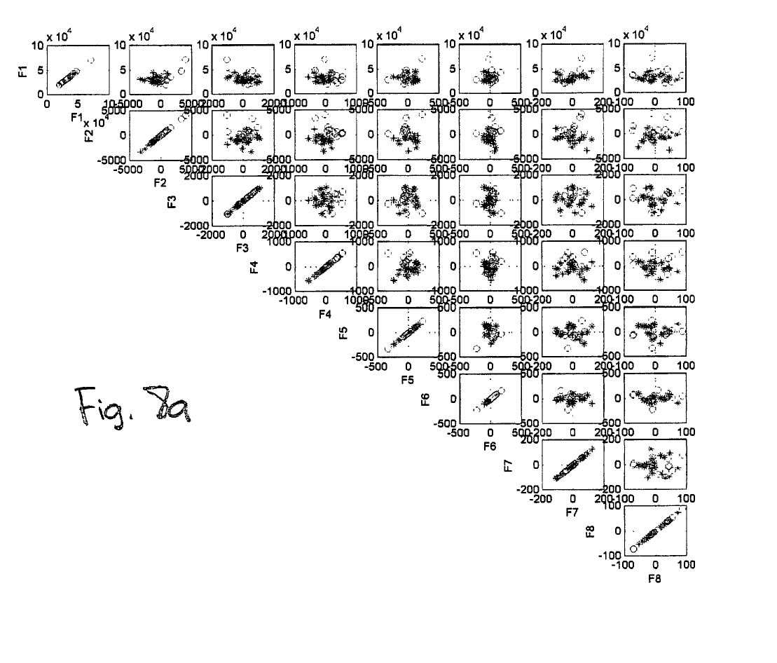

Principle Components Analysis Referring to FIGS. 6a and 6b, respective fluorescence emissions from each of a set of 19 negative control samples and 12 HCV positive control samples were obtained according to the following procedure. Recently received (less than three months frozen storage) plasma samples obtained from newly diagnosed HCV infected patients and uninfected volunteers were thawed for extraction with 50% (v/v) isopropyl alcohol. Over a period of three days a total of 19 negative and 12 positive samples were extracted and the supernate irradiated with 355 nm light for fluorescence measurement. Fluorescence emission was collected between 360 and 585 nm. Fluorescence emissions in FIG. 6a have been point normalized. Fluorescence emissions in FIG. 6b are un-normalized. Each set of fluorescence emissions represents control data that may be used to determine the presence of HCV in a sample.

The fluorescence emissions presented here have had the Raman peaks of water and isopropyl alcohol removed by curve smoothing for clarity. Similar discriminatory results have been obtained without subtraction of water or isopropyl alcohol Raman peaks (approx. 410 and 395 nm respectively).

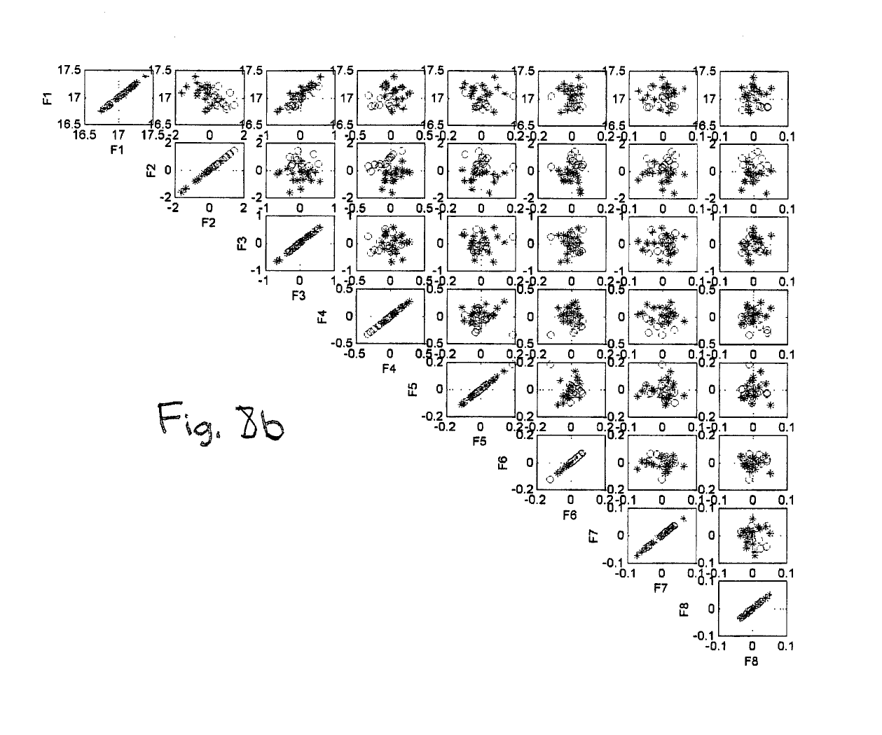

Referring to FIGS. 7a and 7b, respectively, principle components analysis (PCA) was used to find the eight most significant principle components required to explain the variance of the normalized and un-normalized control data of FIGS. 6a and 6b, respectively. Using a dimension reduction algorithm such as principle components analysis to find the principle components of a control data set is equivalent to reducing the dimensionality of the control data set. The dimensionality is reduced because each of the 27 fluorescence emissions of the control data sets of FIGS. 6a and 6b may be essentially reproduced by a weighted sum of the 8 principle components in FIGS. 7a and 7b, respectively. Thus, each principal component obtained from the PCA analysis may be defined as a reduced dimension component of the respective control data set.

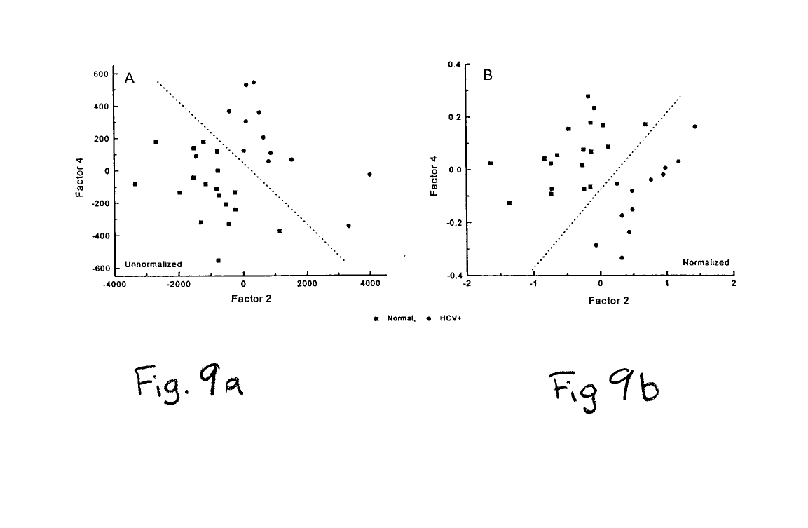

Referring to FIGS. 8a and 8b, fluorescence emissions obtained from individual samples were projected onto the principle components obtained from the PCA analysis of the normalized and un-normalized spectra. Mathematically, projecting a fluorescence emission onto a principle component may be equivalent to determining the dot product of the fluorescence emission and principle component. As defined herein, the projection of a principle component and a fluorescence emission from a sample onto one another is a comparison of a control parameter and a parameter of the fluorescence emission. The results from the projections are presented as two dimensional arrays of factors in which HCV positive samples were assigned a different symbol (circle) than uninfected samples (stars) shown in FIGS. 8a and 8b.

PCA analysis of un-normalized spectra showed that PCA Factors 2 and 4 individually held power to partially distinguish the HCV positive (red circles) and negative (blue asterisks) samples tested (FIG. 8a) as illustrated by the differential distribution of samples along the diagonal axis when compared against themselves (e.g. F2×F4 frame, FIG. 8a). In figures illustrating spectral distributions in 8-factor space, red circles represent HCV positive plasma samples and blue asterisks represent normal samples.

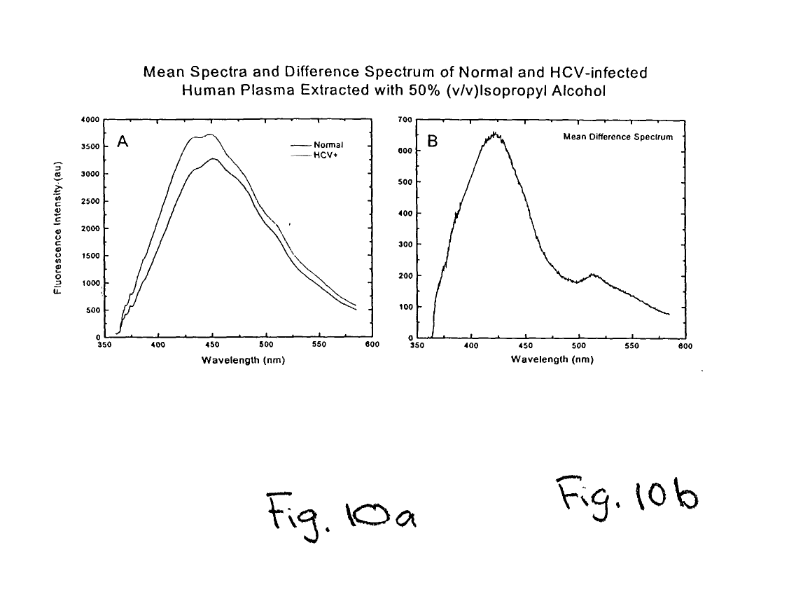

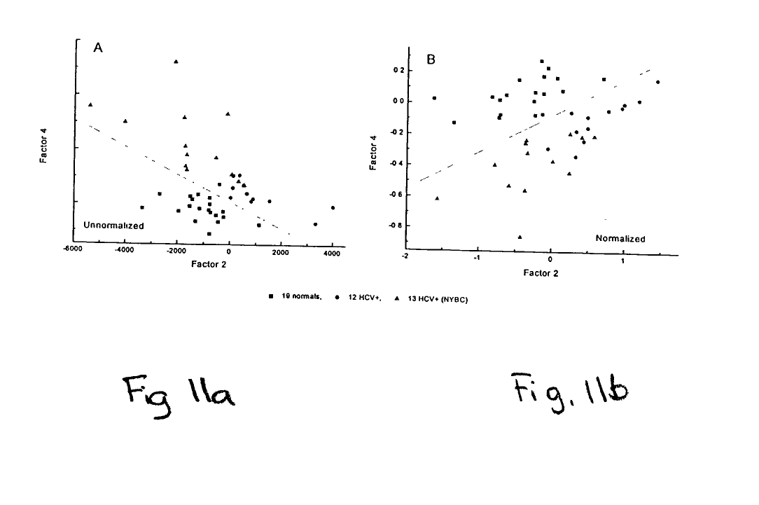

Referring to FIGS. 9a and 9b, factors 2 and 4, when combined, such as by plotted them against one another, fully discriminate between the 19 negative and 12 HCV positive samples in this test group. Each point in the plots of FIG. 9a is a parameter that represents the result of projecting a particular sample emission onto factor 4 (F4 in FIG. 7a) and the result of projecting the same sample emission onto factor 2 (F2 in FIG. 7a). The straight lines in each plot indicate a threshold value parameter for determining whether a sample is from an infected or non-infected sample.

A comparison of a parameter derived from an sample of unknown infection status is accomplished by determining whether the projection from the unknown sample is grouped with the infected or non-infected samples. In FIG. 9a, for example, a point falling above the line indicates a positive sample whereas a point falling below the line indicates a negative sample. Analysis of component vectors derived from the PCA analysis (FIG. 7a) suggests that spectral differences around 400 nm and 475 nm (F2), and 380 nm, 430 nm and 475 nm (F4) provide the majority of the information differentiating infected from uninfected plasma.

Similar results were obtained by PCA analysis of spectra that were normalized to the intensity at 460 nm within each spectrum. Inspection of the eight major factors identified by PCA (FIG. 7b) revealed that Factors 2 and 4 held the greatest discriminatory power for this set. The combination of these two factors also provided full discrimination between the 19 control and 12 HCV-infected samples (FIGS. 8b and 9b) with the individual factors (FIG. 7b) presenting similar, though inverted in some factors, spectral signals to those of the unnormalized set.

The consistency of the results between the normalized and unnormalized data sets, with detection of common discriminating vectors, indicate that the discriminatory power of this analysis is independent of signal intensity and focused on differences in relative levels of groups of spectral features between the infected and uninfected spectra. Comparison of the mean (average) spectra (FIG. 10a) for this sample set revealed notable spectral profile differences consistent with the significant discriminatory PCA vectors, and a mean difference spectrum (enlarged FIG. 10b) with a distinct maximum near 425 nm. The discriminator functions discussed above preferably take advantage of these average differences between emission derived from infected and infection free samples.

### Example 3

A series of relatively recently drawn (1-3 months storage) frozen HCV-positive plasma samples were obtained from a commercial blood bank. These samples were taken from plasma bags (all treated with CPD citrate) derived from fractionated whole blood donations, and were confirmed positive by repeat reactive EIA results or NAT testing. These samples therefore are representative of asymptomatic donors except that plasma derived from fractionated blood will contain an additional 20-30% (v/v) diluent as compared to direct vacutainer-drawn samples.

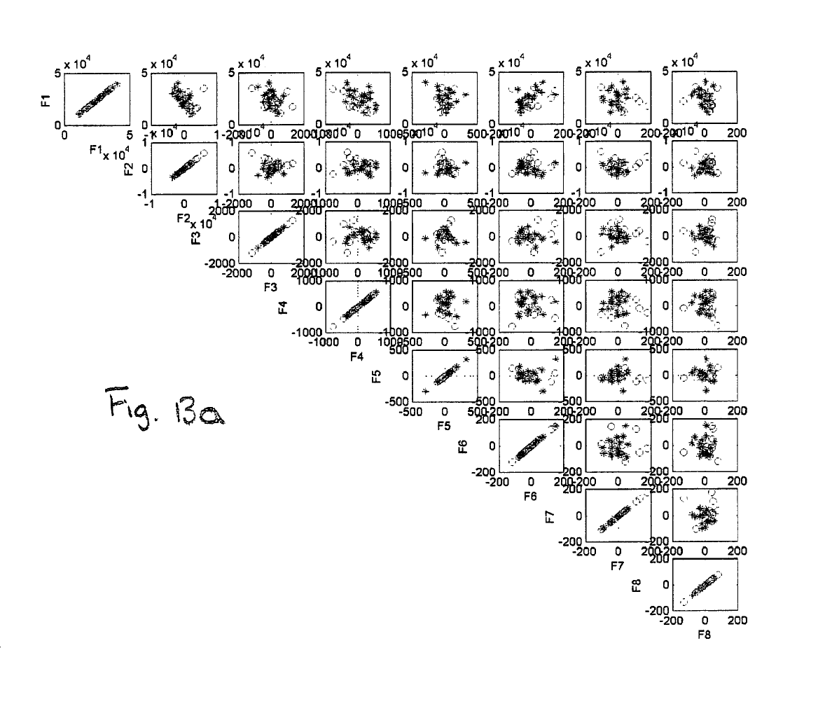

Thirteen blood bank samples were processed by isopropyl alcohol extraction, and fluorescence spectra were measured at 355 nm excitation. The features of the observed spectra were similar to those previously obtained with positive samples. As shown in FIGS. 11a and 11b, when the fluorescence emission spectra of these samples were projected onto the vectors identified by the PCA of 19 neg/12HCV pos samples described above, all thirteen samples fell on the positive side of the line dividing the positive and negative base samples as defined by Factors 2 and 4. Similar results were obtained with both normalized (FIG. 11a) and unnormalized (FIG. 11b) spectra and respective PCA vectors. In these figures, blue squares represent the negative samples, and red circles represent positive samples forming the 19N/12P database, while green triangles represent the tested HCV-positive samples from the blood bank.

The blood bank samples, however, extended the distribution of positive samples along the projection, suggesting the existence of a process-dependent spectral feature (other than intensity) that may be accommodated by the PCA discrimination method employing Factors 2 and 4, and does not alter the scoring of diluted samples. Sample acquisition from fractionated plasma for preclinical, clinical and blood bank testing may therefore be acceptable. All known HCV-positive plasma samples have tested positive by 50% (v/v) isopropyl alcohol extraction and fluorescence spectral analysis with 355 nm laser excitation.

### Example 4

A series of relatively recently drawn (1-3 months storage) frozen HIV-positive plasma samples were obtained from newly diagnosed patients who had not yet received anti-viral therapy, and were collected directly into both EDTA and citrate vacutainer tubes for plasma preparation. Aliquots of the citrated samples were extracted with 50% IPA and subjected to fluorescence analysis as previously described.

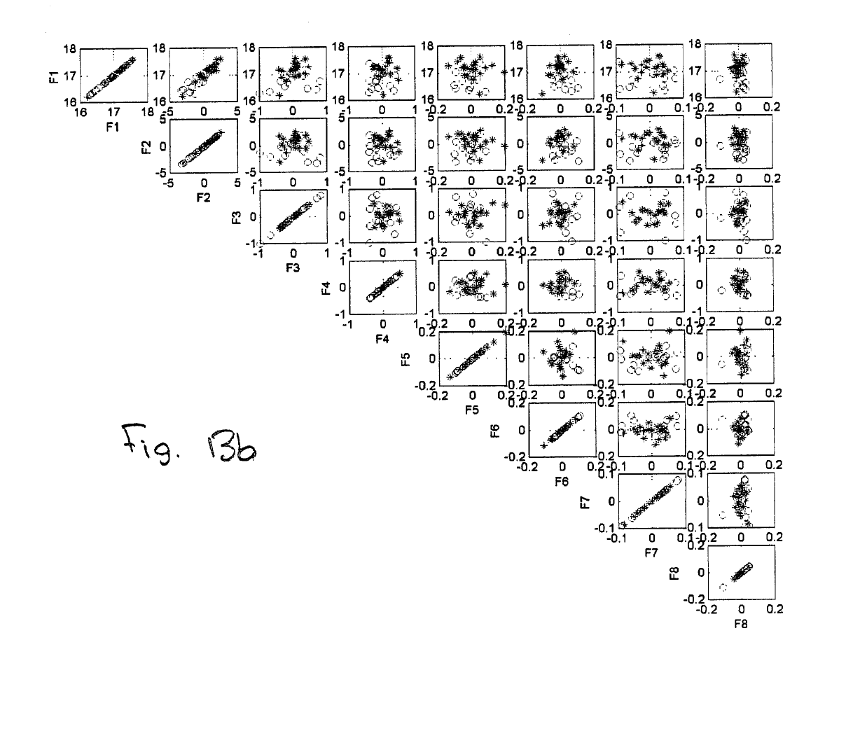

When the resulting spectra were projected onto PCA Factors 2 and 4 as described above, all HCV positve plasma samples tested negative (FIGS. 12a and 12b), suggesting that the observed spectral differences were highly specific for HCV infection and hold substantial discriminatory power.

### Example 5

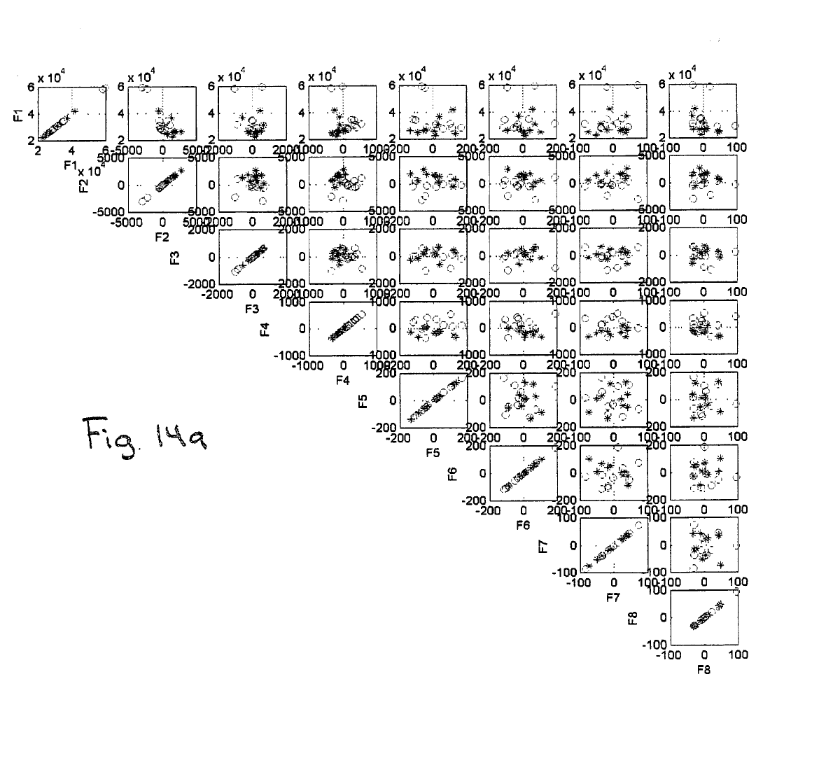

The archived data obtained with PEG fractionation of the EDTA plasma samples of the same 19 negative/12 HCV positive donors used for 50% IPA PCA analysis above were compiled and subjected to similar analysis. The results of this assessment showed that the spectra obtained from the PEG pellets did not fully discriminate between infected and control groups in either normalized or unnormalized data sets with any two dimensional combination of vectors (FIGS. 13a,b).

Based on the ability of the 50% (v/v) IPA extracted spectra to provide full discrimination in two well defined dimensions for HCV, it is expected to have significantly enhanced utility compared to the PEG/EDTA method. Although no complete two dimensional discrimination was noted with the PEG pellet data set, one embodiment of the invention includes combining projections of sample emission along more than two factors to increase discrimination. It must be noted that different plasma anticoagulants were used in the PEG (EDTA) and 50% IPA (citrate) methods which might contribute to the difference in the discriminatory power noted.

### Example 6

A similar study directly comparing the 50% IPA and PEG precipitation methods with the same donor sample set (except for anticoagulant) on the same day, was conducted. Ten recent positive and ten negative donors for which sufficient plasma of both types was available were treated and their fluorescence assessed according to standard procedures. PCA analysis of the two data sets provided separate sets of principal components.

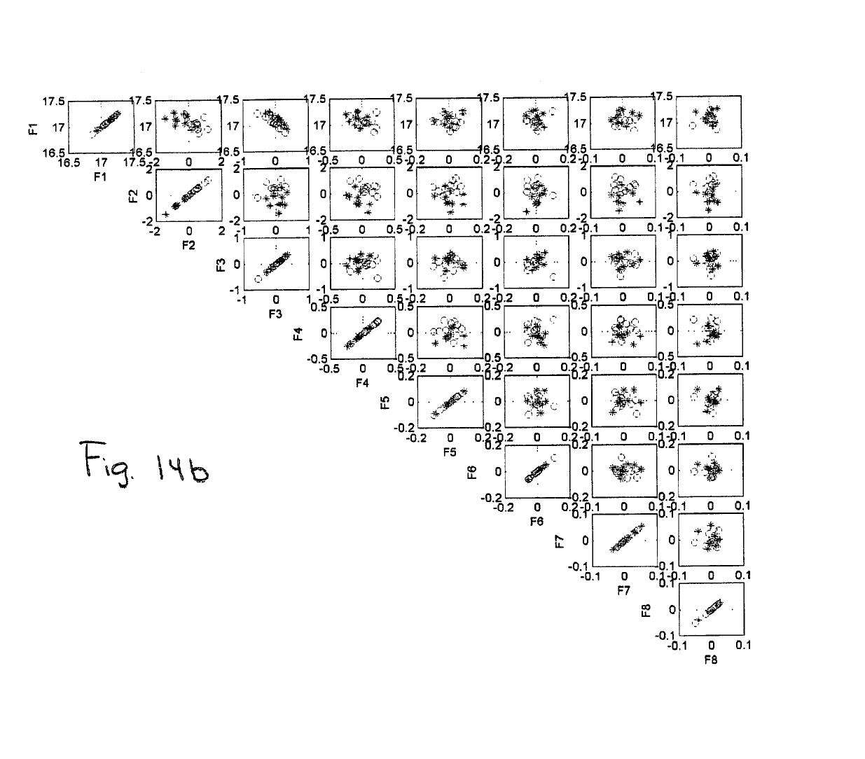

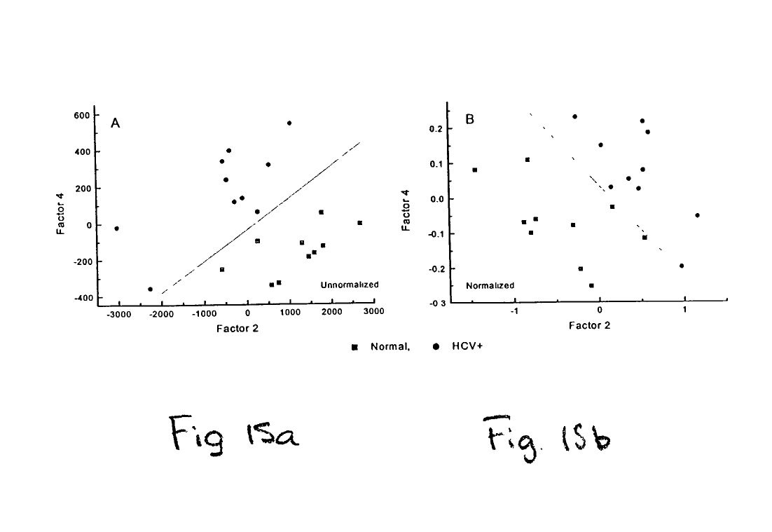

When positive and negative samples were unmasked, the data set obtained by 50% isopropyl alcohol extraction showed complete discrimination of the groups in two dimensions (FIGS. 14a,b), with Factors 2 and 4, as seen previously, achieved full separation of positive and negative subsets (FIGS. 15a,b).

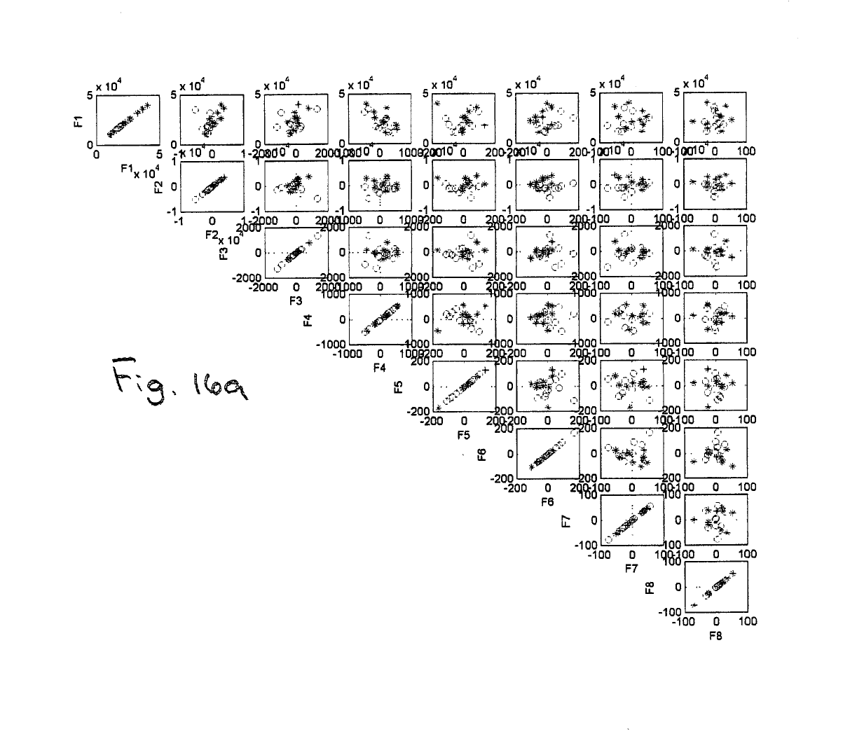

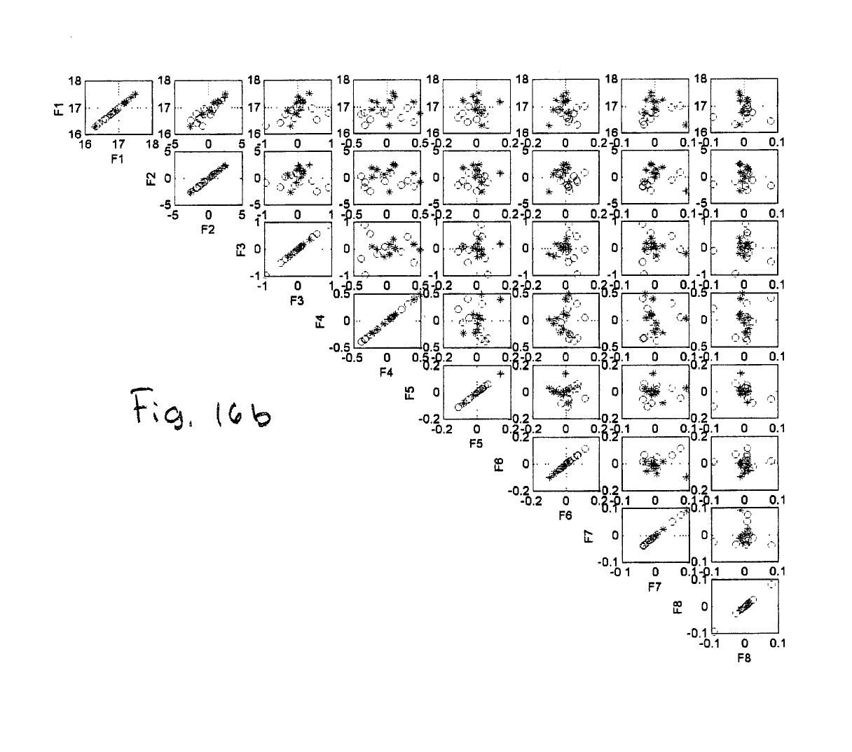

PCA analysis of the PEG spectra did not identify any two dimensional vector combinations that provided complete discrimination (FIGS. 16a,b) between HCV-infected and normal plasma. The combination of factors 2 and 6 provided the most significant partial discrimination of these data sets (FIGS. 17a,b).

Comparison of these results indicated that for 2-dimensional PCA discrimination of HCV-positive and control samples, 50% (v/v) isopropyl alcohol extraction provides greater power than the PEG precipitation method. Consistent with visual comparison, univariate statistical analysis confirmed that vector 2 partial discrimination was statistically significant for both the IPA and PEG methods (p=0.0031 and p=0.0084 respectively), whereas, multivariate analysis of the 8 major vectors showed the overall PEG resolution to be marginal (p=0.0763) while the IPA complete discrimination was highly significant (p<0.0001).

To assess whether the spectral features associated with discrimination by 50% (v/v) IPA extraction were dependent on the anticoagulant used to prepare the plasma sample, a set of five HCV positive and 5 HCV negative plasma donors from whom both EDTA and citrate plasma were available, were extracted by the standard IPA procedure.

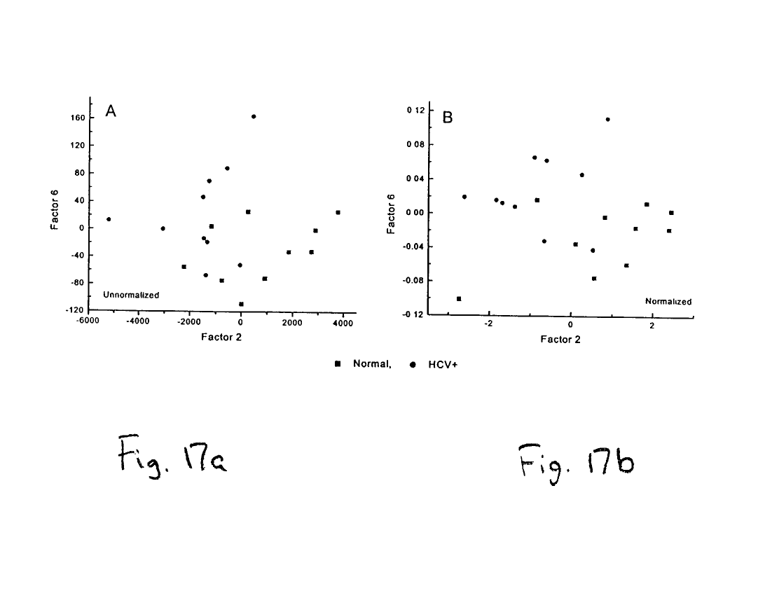

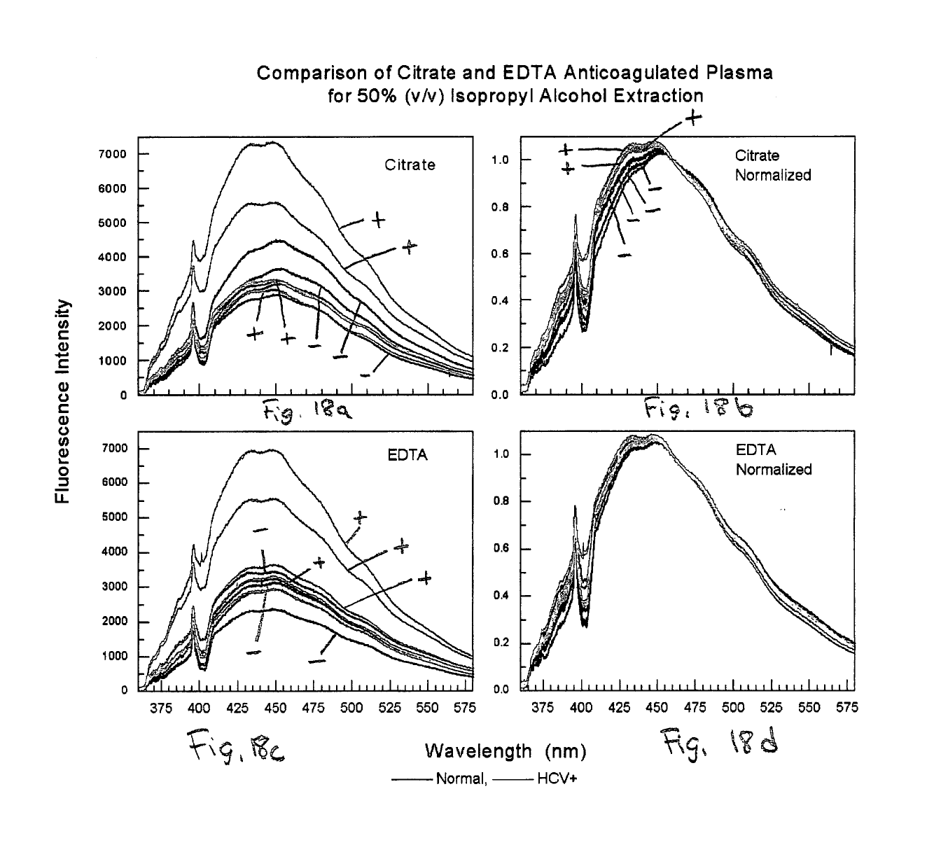

Visual differentiation between the control and HCV-infected spectra was clear in the citrate anticoagulated group but minimal in the EDTA anticoagulated group (FIGS. 18a-18d). Normalized spectra revealed significantly different proportions of the main peak and blue shoulder in citrated control and infected groups that were absent in EDTA anticoagulated samples. Based on these results, samples fractionated using organic liquid, such as via 50% IPA extraction, are preferably standardized with citrate anticoagulated samples.

While the above invention has been described with reference to certain preferred embodiments, it should be kept in mind that the scope of the present invention is not limited to these. Thus, one skilled in the art may find variations of these preferred embodiments which, nevertheless, fall within the spirit of the present invention, whose scope is defined by the claims set forth below.

What is claimed is:

1. A method for determining whether a biological sample is infected with HIV, comprising:
obtaining a fluorescence emission from a mixture obtained by contacting the biological sample with an organic polymer, at least a portion of the biological sample being soluble in the organic polymer, the fluorescence emission being essentially free of fluorescence resulting from substances not-native to the organism; and
comparing at least one parameter of the fluorescence emission with at least one corresponding control parameter to determine whether the biological sample is infected with HIV.

2. The method of claim 1, wherein the organic polymer comprises at least one of a poly-alcohol, a poly-ether, heparin, and dextran sulfate.

3. The method of claim 1, wherein the organic polymer comprises at least one polyethylene glycol.

4. The method of claim 1, wherein comparing the at least one parameter and the at least one control parameter comprises using a dimension reduction algorithm.

5. The method of claim 4, wherein the dimension reduction algorithm comprises at least one of principal component analysis, factor analysis, principal component regression, and singular value decomposition.

6. The method of claim 1, wherein the biological sample is one of blood, plasma, or combination thereof.

7. A method for determining whether a biological sample is infected with hepatitis, comprising:
obtaining a fluorescence emission from a mixture obtained by contacting the biological sample with an organic polymer, at least a portion of the biological sample being soluble in the organic polymer, the fluorescence emission being essentially free of fluorescence resulting from substances not-native to the organism; and
comparing at least one parameter of the fluorescence emission with at least one corresponding control parameter to determine whether the biological sample is infected with hepatitis.

8. The method of claim 7, wherein the organic polymer comprises at least one of a poly-alcohol, a poly-ether, heparin, and dextran sulfate.

9. The method of claim 7, wherein the organic polymer comprises at least one polyethylene glycol.

10. The method of claim 7, wherein comparing the at least one parameter and the at least one control parameter comprises using a dimension reduction algorithm.

11. The method of claim 10, wherein the dimension reduction algorithm comprises at least one of principal component analysis, factor analysis, principal component regression, and singular value decomposition.

12. The method of claim 7, wherein the biological sample is one of blood, plasma, or combination thereof.

13. A method for processing a biological sample to determine whether the biological sample is infected with HIV, the sample having been contacted with at least one of a poly-alcohol, a poly-ether, heparin, phosphotungstic acid, and dextran sulfate to thereby fractionate the sample into first and second fractions, the first fraction including a greater relative amount of lipoprotein than the second fraction, the method comprising:
obtaining a fluorescence emission from the first fraction, the fluorescence emission being essentially free of fluorescence resulting from substances not-native to the organism; and
comparing at least one parameter of the fluorescence emission with at least one corresponding control parameter to determine whether the biological sample is infected with HIV.

14. The method of claim 13, wherein the sample has been contacted with a polyethylene glycol.

15. A method for processing a biological sample to determine whether the biological sample is infected with hepatitis, the sample having been contacted with at least one of a poly-alcohol, a poly-ether, heparin, phosphotungstic acid, and dextran sulfate to thereby fractionate the sample into first and second fractions, the first fraction including a greater relative amount of lipoprotein than the second fraction, the method comprising:
obtaining a fluorescence emission from the first fraction, the fluorescence emission being essentially free of fluorescence resulting from substances not-native to the organism; and
comparing at least one parameter of the fluorescence emission with at least one corresponding control parameter to determine whether the biological sample is infected with hepatitis.

16. The method of claim 15, wherein the sample has been contacted with a polyethylene glycol.

17. A method for determining whether a biological sample is infected with HIV, comprising:
obtaining a fluorescence emission from a mixture obtained by contacting the biological sample with at least one organic compound selected from the group consisting of alcohols, ketones, nitrites, and aldehydes, the fluorescence emission being essentially free of fluorescence resulting from substances not-native to the organism; and
comparing at least one parameter of the fluorescence emission with at least one corresponding control parameter to determine whether the biological sample is infected with HIV.

18. The method of claim 17, wherein the organic compound includes fewer than about 11 carbon atoms.

19. The method of claim 18, wherein the organic compound is acetonitrile.

20. The method of claim 18, wherein the organic compound is iso-propyl alcohol.

21. The method of claim 17, wherein the mixture includes a supernate and the fluorescence emission is obtained from the supernate.

22. The method of claim 17, wherein the biological sample comprises plasma.

23. A method for determining whether a biological sample is infected with hepatitis, comprising:
obtaining a fluorescence emission from a mixture obtained by contacting the biological sample with at least one organic compound selected from the group consisting of alcohols, ketones, nitrites, and aldehydes, the fluorescence emission being essentially free of fluorescence resulting from substances not-native to the organism; and
comparing at least one parameter of the fluorescence emission with at least one corresponding control parameter to determine whether the biological sample is infected with hepatitis.

24. The method of claim 23, wherein the organic compound includes fewer than about 11 carbon atoms.

25. The method of claim 24, wherein the organic compound is acetonitrile.

26. The method of claim 24, wherein the organic compound is iso-propyl alcohol.

27. The method of claim 23, wherein the mixture includes a supernate and the fluorescence emission is obtained from the supernate.

28. The method of claim 23, wherein the biological sample comprises plasma.

29. A method for determining whether a biological sample is infected with HIV, comprising:
obtaining fluorescence data from fluorescence emission emitted from the biological sample, the fluorescence emission being essentially free of fluorescence resulting from substances not-native to the organism;
reducing a dimensionality of the fluorescence data to prepare reduced dimension data having at least one parameter; and
comparing at least one parameter of the reduced dimension data with at least one corresponding control parameter to determine whether the biological sample is infected with HIV.

30. The method of claim 29, wherein reducing the dimensionality comprises subjecting the fluorescence data to principal component analysis, factor analysis, principal component regression, or singular value decomposition.

31. A method for determining whether a biological sample obtained from a mammal is infected with HIV, comprising:
obtaining a sample fluorescence emission from the biological sample, the sample fluorescence emission being essentially free of fluorescence resulting from substances not-native to the mammal;
providing control data comprising fluorescence emissions obtained from a plurality of control samples;
reducing a dimensionality of the control data to thereby prepare a reduced dimension component; and
projecting at least a portion of the sample fluorescence emission onto the reduced dimension component to determine whether the biological sample is infected with HIV.

32. The method of claim 31, wherein the reduced dimension component is a principle component and reducing a dimensionality comprises subjecting the control data to a principle components algorithm.

33. A method for determining whether a biological sample obtained from a mammal is infected with hepatitis, comprising:
obtaining a sample fluorescence emission from the biological sample, the sample fluorescence emission being essentially free of fluorescence resulting from substances not-native to the mammal;
providing control data comprising fluorescence emissions obtained from a plurality of control samples;
reducing a dimensionality of the control data to thereby prepare a reduced dimension component; and
projecting at least a portion of the sample fluorescence emission onto the reduced dimension component to determine whether the biological sample is infected with hepatitis.

34. The method of claim 33, wherein the reduced dimension component is a principle component and reducing a dimensionality comprises subjecting the control data to principle components algorithm.

36. A method for determining whether a biological sample is infected with hepatitis, comprising:
obtaining fluorescence data from fluorescence emission emitted from the biological sample, the fluorescence emission being essentially free of fluorescence resulting from substances not-native to the organism;
reducing a dimensionality of the fluorescence data to prepare reduced dimension data having at least one parameter; and
comparing at least one parameter of the reduced dimension data with at least one corresponding control parameter to determine whether the biological sample is infected with hepatitis.

37. The method of claim 36, wherein reducing the dimensionality comprises subjecting the fluorescence data to principal component analysis, factor analysis, principal component regression, or singular value decomposition.
